SEQUENCE LISTING (7 sequences)

SEQ ID NO: 1 - protein, 92 aa, Homo sapiens
MSELEKAMVALIDVFHQYSGREGDKHKLKKSELKELINNELSHFLEEIKEQEVVDKVMETLDNDGDGECDFQEFMAFVAMVTTACHEFFEHE

SEQ ID NO: 2 - protein, 97 aa, Homo sapiens
MSELEKAMVALIDVFHQYSGREGDKHKLKKSELKELINNELSHFLEEIKEQEVVDKVMETLDNDGDGECDFQEFMAFVAMRRSCKKADSKGLQPSRS

SEQ ID NO: 3 - protein, 25 aa, Homo sapiens
MSELEKAMRRSCKKADSKGLQPSRS

SEQ ID NO: 4 - protein, 54 aa, Homo sapiens
MSELEKAMEIKEQEVVDKVMETLDNDGDGECDFQEFMAFVAMVTTACHEFFEHE

SEQ ID NO: 5 - protein, 59 aa, Homo sapiens
MSELEKAMEIKEQEVVDKVMETLDNDGDGECDFQEFMAFVAMRRSCKKADSKGLQPSRS

SEQ ID NO: 6 - protein, 69 aa, Homo sapiens
MSELEKAMVALIDVFHQYSGREGDKHKLKKSELKELINNELSHFLEEIKEQERRSCKKADSKGLQPSRS

SEQ ID NO: 7 - protein, 17 aa, Homo sapiens
RRSCKKADSKGLQPSRS

(19) United States
(12) Patent Application Publication
(10) Pub. No.: US 2017/0176460 A1
(43) Pub. Date: Jun. 22, 2017

(54) NEURAL SPECIFIC S100-BETA FOR BIOMARKER ASSAYS AND DEVICES FOR DETECTION OF A NEUROLOGICAL CONDITION

(71) Applicant: BANYAN BIOMARKERS, INC., Alachua, FL (US)

(72) Inventor: Stephen LARNER, Newberry, FL (US)

(73) Assignee: BANYAN BIOMARKERS, INC., Alachua, FL (US)

(21) Appl. No.: 15/444,213

(22) Filed: Feb. 27, 2017

Related U.S. Application Data
(63) Continuation of application No. 14/293,758, filed on Jun. 2, 2014.
(60) Provisional application No. 61/829,249, filed on May 31, 2013.

(51) Int. Cl. G01N 33/68; G01N 33/543

(52) U.S. Cl. CPC G01N 33/6896; G01N 33/54386; G01N 2800/2821; G01N 2800/2835; G01N 2800/2857; G01N 2800/2871; G01N 2333/916; G01N 2333/988

(57) ABSTRACT

An in vitro diagnostic (IVD) device is used to detect the presence of and/or severity of neural injuries or neuronal disorders in a subject. The IVD device relies on an immunoassay which identifies biomarkers that are diagnostic of neural injury and/or neuronal disorders in a biological sample, such as whole blood, plasma, serum, and/or cerebrospinal fluid (CSF). An IVD device may measure one or more of several neural specific markers in a biological sample and output the results to a machine readable format, either to a display device or to a storage device internal or external to the IVD.

### CROSS-REFERENCE TO RELATED APPLICATIONS

This application is a continuation of U.S. patent application Ser. No. 14/293,758 filed Jun. 2, 2014, which claims priority to U.S. Provisional Patent Application No. 61/829,249 filed on May 31, 2013. Each related application is herein incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 750582001901_SeqList.txt, date recorded: Feb. 27, 2017, size: 4,455 bytes).

### FIELD OF THE INVENTION

The invention provides for compositions of matter, methods, processes, kits, and in vitro diagnostic devices which enables the reliable detection of damage to the nervous system (central nervous system (CNS) and peripheral nervous system (PNS)), brain injury, and neural disorders of an individual through biomarker identification.

### BACKGROUND OF THE INVENTION

The field of clinical neurology remains frustrated by the recognition that secondary injury to a CNS tissue associated with physiologic response to the initial insult could be lessened if only the initial insult could be rapidly diagnosed or, in the case of a progressive disorder, before stress on CNS tissues reached a preselected threshold. Traumatic, ischemic, and neurotoxic chemical insult, along with generic disorders, all present the prospect of brain damage. Traumatic, ischemic, and neurotoxic chemical insult also present the prospect of brain or other neurological damage.

One of many neurological conditions, traumatic brain injury (TBI), occurs when external forces, through direct impact or acceleration, traumatically injure the brain. This often occurs through falls, vehicle accidents, and violence. TBI can be characterized by its severity, from mild to moderate to severe, and the effects of such injury can be physical, cognitive, social, emotional, or behavioral and clinical outcomes range from complete recovery to permanent disability and death. Post-traumatic stress disorder (PTSD) is a psychological trauma that mirrors the symptoms of more moderate TBI, thereby making both injuries hard to distinguish and diagnose. Moreover, because of the similar symptoms of several other neurological conditions, such as stroke, subarachnoid hemorrhage, or a neurodegenerative disease, distinguishing one injury type from another has frustrated clinicians for years.

More common methods and areas of continuing research for diagnosing a neurological condition usually involve a neurological examination and the assignment of a Glasgow Coma Score (GCS) to an individual. These methods are of limited value and often preclude a nuanced diagnosis due to the subjectivity of the testing, and the ability of a patient to knowingly alter their true response to achieve a desired result. Neuroimaging, such as computed tomography (CT) and magnetic resonance imaging (MRI), are also widely used to help determine the scope of injury and potential for intervention. However, these tests are both costly and time consuming, as well as are frustrated by the same problems of having an inability to distinguish one injury type from another. In addition, individuals with only mild or moderate TBI may be unaware damage has occurred and fail to seek treatment. It should be appreciated that repeated mild to moderate injuries can have a cumulative effect and result overall in a poor clinical diagnosis.

Early clinical diagnosis of a neurological condition continues to be an area requiring further development. Early diagnosis can limit the damage caused by facilitating earlier intervention. Much emphasis is being placed on developing biomarkers as early indicators of CNS damage. Upon injury to neural cells, neural proteins are released into the interstitial fluid of the brain and eventually cross the blood brain barrier where they can be easily measured. Identifying specific proteins and measuring the levels that enter circulation upon the onset of a neurological condition can provide an effective early means of detecting the severity and type of the injury.

A number of biomarkers have been identified as being associated with diagnosing several neurological conditions or nerve cell damage. These biomarkers included spectrin breakdown products such as SBDP150, SBDP150i, SBDP145 (calpain mediated acute neural necrosis), SBDP120 (caspase mediated delayed neural apoptosis), UCH L1 (neuronal cell body damage marker), and MAP2 dendritic cell injury associated marker. The nature of these biomarkers is detailed in U.S. Pat. Nos. 7,291,710 and 7,396,654, the contents of which are hereby incorporated by reference. Other biomarkers may be used to detect for neural injury, neuronal disease, or neural disorders, detailed in the disclosures presented in US 2007/0003982 A1, US 2005/0260697 A1, US 2009/0317805 A1, US 2005/0260654 A1, US 2009/0087868 A1, US 2010/0317041 A1, US 2011/0177974 A1, US 2010/0047817 A1, US 2012/0196307 A1, US 2011/0082203 A1, US 2011/0097392 A1, US 2011/0143375 A1, US 2013/0029859 A1, US 2012/0202231 A1, and US 2013/0022982 A1, the contents of which are also hereby incorporated by reference.

S100β is abundantly expressed in astrocytes and is commonly used as an astrocytic marker in studies of the mammalian CNS. As such, S100β has been widely used as a biomarker of a variety of neurological conditions and it is elevated upon damage to neurons at higher levels than most other neuronal specific markers, making it an easy protein to assay. Conventional S100β is known to be an 11 kDa dimeric calcium binding protein, which is highly expressed in astroglial and oligodendroglial cells. However, using elevated levels of S100β in serum or CSF to diagnose a neurological condition does not always reliably correlate with clinical outcome. This may be due to the fact that S100β is expressed in several non-neuronal tissues, including adipose, skeletal muscle, cardiac, chondrocytes, and epidermal cells. Furthermore, circulating levels of S100β are elevated in a number of non-neuronal traumas, such as liver injury, femoral fracture, and myocardial infarction. Therefore, these non-neuronal sources of S100β cannot be excluded as sources of elevation, thus precluding a clinical validation of an S100β diagnostic assay. Additionally, as it relates to TBI, as brain injuries are milder, the smaller increases in S100β levels make testing the increases over basal levels less accurate.

Thus, there is a need to identify the underlying molecular pathology of various neurological conditions in order to classify and distinguish these traumas. There also exists the need to identify a brain specific or brain enriched isoform of S100β to allow for new, neural specific compositions of matter, such as antigens or antibodies, to support processes and assays for providing improved measurement of neuron-specific or neuron enriched biomarkers of a neurological condition, either alone or in synergistic combination, with the specificity and sensitivity necessary to distinguish a neurological condition and to detect mild and moderate forms of brain injury. There also remains an unmet need for clinical intervention through the use of an in vitro diagnostic device to identify these neurochemical markers so that subject results may be obtained rapidly in any medical setting to direct the proper course of treatment for subjects suffering from a neural injury or neuronal disorder.

### SUMMARY OF THE INVENTION

A compositions of matter, antibody and antigen related thereto, are provided, along with methods, processes, kits, and in vitro diagnostic devices specifically designed and calibrated to detect protein markers that are differentially present in the samples of patients suffering from neural injury and/or neuronal disorders including neurotoxicity and nerve cell damage. These devices present a sensitive, quick, and non-invasive method to aid in diagnosis of neural injury and/or neuronal disorders by detecting and determining the amount of biomarkers that are indicative to the respective injury type. The measurement of these markers in patient samples, alone or in combination with other markers for the injury type, provides information that a diagnostician can correlate with a probable diagnosis of the extent of an injury such as sports concussion, traumatic brain injury (TBI), and stroke.

In a particular embodiment, an in vitro diagnostic device is provided to measure biomarkers that are indicative of traumatic brain injury, stroke, Alzheimer's disease, epilepsy, hypoxic ischemic encephalopathy (HIE), chronic traumatic encephalopathy (CTE), neural disorders, brain damage, neural damage due to drug or alcohol addiction, or other diseases and disorders associated with the brain or nervous system, such as the CNS or PNS. In some embodiments, the biomarkers are proteins, fragments, or derivatives thereof, and are associated with neuronal cells, brain cells, or any cell that is present in the brain, CNS, and PNS.

In a particular embodiment, the biomarkers are neural proteins, peptides, fragments, or derivatives thereof, which are detected by an assay. An in vitro diagnostic device is also provided that further includes a process for determining the neurological condition of a subject or cells from the subject, including measuring a sample obtained from the subject or cells from the subject at a first time for a quantity of a first biomarker which represents a brain specific or brain enriched isoform of S100β, namely S100β2, S100β3, S100β4, S100β5, and S100β6. These markers may be measured alone or in combination with other markers such as GFAP, UCH-L1, NSE, conventional S100β (S100β1), MBP, MAP2, SBDP, CRMP, synaptotagmin, or neurensin-1 (p24). Through comparison of the quantity of the first biomarker and the quantity of the at least one additional neuroactive biomarker to normal levels for each biomarker, the neurological condition of the subject is determined over one or more possible injury indication that only one biomarker may represent. An in vitro diagnostic device that, in some embodiments, incorporates an assay for determining the neurological condition of a subject or neural cells from the subject is also provided. The assay includes at least a first biomarker specifically binding agent wherein a first biomarker is a neural specific or brain specific or brain enriched isoform of S100β, namely S100β2, S100β3, S100β4, S100β5, and S100β6. In a particular embodiment an assay is incorporated which may detect one or more additional markers selected from the group of GFAP, UCH-L1, NSE, S100β1, MBP, MAP2, SBDP, CRMP, synaptotagmin, or neurensin-1 (p24).

A device is also provided that uses a process for determining if a subject has suffered mild, moderate, or severe TBI in an event which includes measuring a sample obtained from the subject or cells from the subject at a first time after the event for a quantity of a neural specific or brain specific or brain enriched isoform of S100β, namely S100β2, S100β3, S100β4, S100β5, and S100β6. S100β2, S100β3, S100β4, S100β5, and S100β6 have a particular synergy being measured with GFAP and UCH-L1 for diagnosing different severities of brain injury, as well as distinguishing injury types, such as determining whether a subject is suffering from TBI, stroke, subarachnoid hemorrhage, or other neural disorders and neural degenerative diseases. Thus, by comparing the quantity of the neural specific isoforms of S100β, GFAP, and UCH-L1 to each other and to a metric of the expected level in a non-injured subject, using an algorithm of the assay output and a pre-programmed comparison metric, which has been clinically validated, a device interpolates the data to determine if the subject has suffered an injury (TBI, stroke, SAH, etc.), the severity of the injury (mild, moderate, or severe), and, by including even more markers, may determine the time after injury and predict a recovery outcome. A comparison of these markers may also be used to determine other neurological disorders such as Alzheimer's and Parkinson's disease, and may predict other neural injuries using this or any number of additional biomarkers, such as neurotoxicity such as is disclosed in WO/2011/123844 and whose disclosure is incorporated herein by reference.

In a particular embodiment, a kit is provided for analyzing cell damage in a subject. The kit, that in particular embodiments, includes: (a) a substrate for holding a biological sample isolated from a human subject suspected of having a damaged nerve cell; (b) an agent that specifically binds at least one or more of the neural proteins; and (c) printed instructions for reacting the agent with the biological sample or a portion of the biological sample to detect the presence or amount of at least one marker in the biological sample. The agents accompanying such kits are those reagents, antigens, antibodies, and/or recombinant proteins for the brain specific or brain enriched isoform of S100β, namely S100β2, S100β3, S100β4, S100β5, and S100β6. The kits may further include reagents, antigens, antibodies, and/or recombinant proteins for an additional one or more neuroactive biomarker, such as glial fibrillary acidic protein (GFAP), ubiquitin carboxyl-terminal esterase L1 (UCH-L1), Neuron specific enolase (NSE), spectrin breakdown products (SBDP), that in particular inventive embodiments are SBDP150, SBDP150i SBDP145, SBDP120, S100β1,), microtubule associated proteins (MAP), that in particular inventive embodiments are MAP2, MAP1, MAP3, MAP4, MAP5, myelin basic protein (MBP), Tau, Neurofilament protein (NF), Cannabinoid Receptor (CB), CAM proteins, Synaptic protein, collapsin response mediator proteins (CRMP), inducible nitric oxide synthase (iNOS), Neuronal Nuclei protein (NeuN), 2′,3′-cyclic nucleotide-3′-phosphohydrolase (CNPase), Neuroserpin, alpha-internexin, microtubule-associated protein 1 light chain 3 (LC3), Neurofascin, the glutamate transporters (EAAT), Nestin, Cortin-1, 2′, and BIII-Tubulin

The biological sample in some embodiments is a fluid in communication with the nervous system of the subject prior to being isolated from the subject (e.g., CSF or blood), and the agent can be an antibody, aptamer, or other molecule that specifically binds at least one or more of the neural proteins. The kit can also include a detectable label such as one conjugated to the agent, or one conjugated to a substance that specifically binds to the agent (e.g., a secondary antibody).

In a particular embodiment, a composition of matter, an antigenic polypeptide fragment, antigens, and antibodies are provided with the antibodies being raised against a peptide having the sequence of SEQ ID NO 2; SEQ ID NO 3; SEQ ID NO 4; SEQ ID NO 5; and SEQ ID NO 6, which represent the S100β isoforms, S100β2-S100β6, respectively.

In a particular embodiment, a diagnostic processes and methods are provided in order to diagnose a neural injury or neuronal disorder. The diagnostic methods make use of the proteins, antigens, and antibodies raised against a peptide having the sequence of SEQ ID NO 2; SEQ ID NO 3; SEQ ID NO 4; SEQ ID NO 5; and SEQ ID NO 6. In addition, one or more additional biomarkers may be measured. These methods or processes may be incorporated into an assay, or into a cartridge containing an assay and reagents to be used to detect the markers herein described, alone, or in combination.

Other aspects of the invention are described infra.

### BRIEF DESCRIPTION OF THE DRAWINGS

FIG. 1 are bar graphs showing the concentration of UCH-L1 and GFAP as well as a biomarker not selected for diagnosis of neurological condition, S100β, provided as a function of injury magnitude between control, mild, and moderate traumatic brain injury;

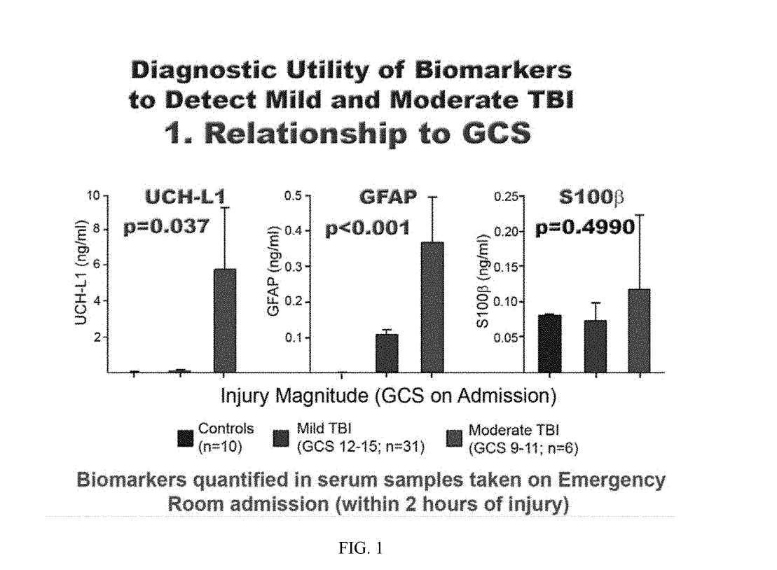

FIG. 2 are bar graphs showing the concentration of the same markers as depicted in FIG. 1 with respect to initial evidence upon hospital admission as to lesions in tomography scans;

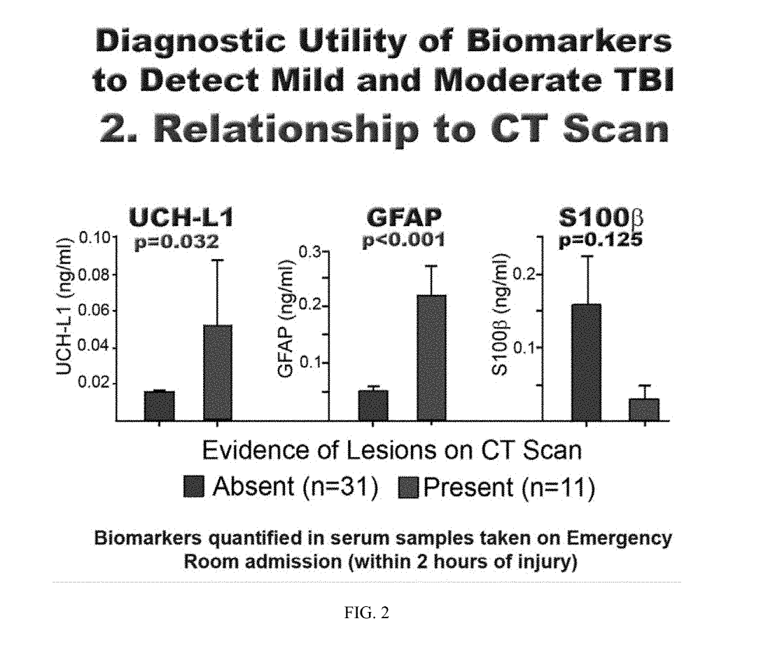

FIG. 3 represents biomarker levels in human subjects with varying types of brain injury;

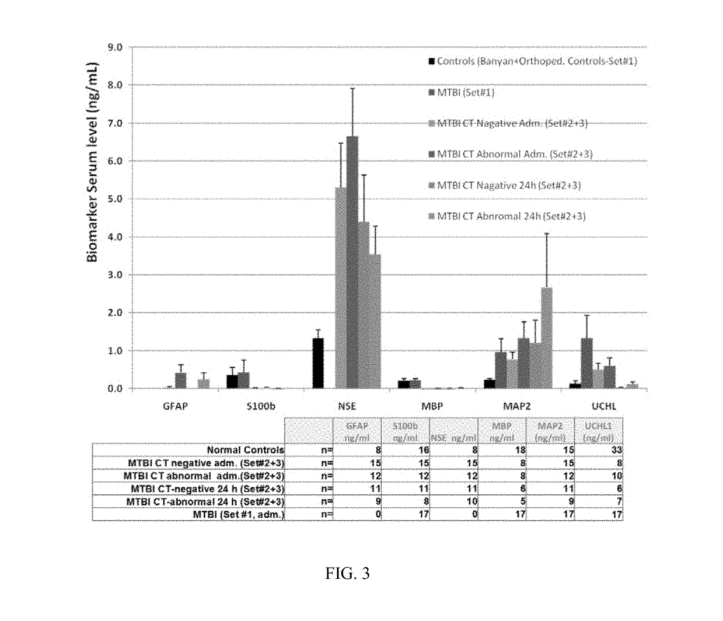

FIG. 4 represents an ROC curve showing the improved specificity and synergistic effects for a neurological condition by using a combination of biomarkers;

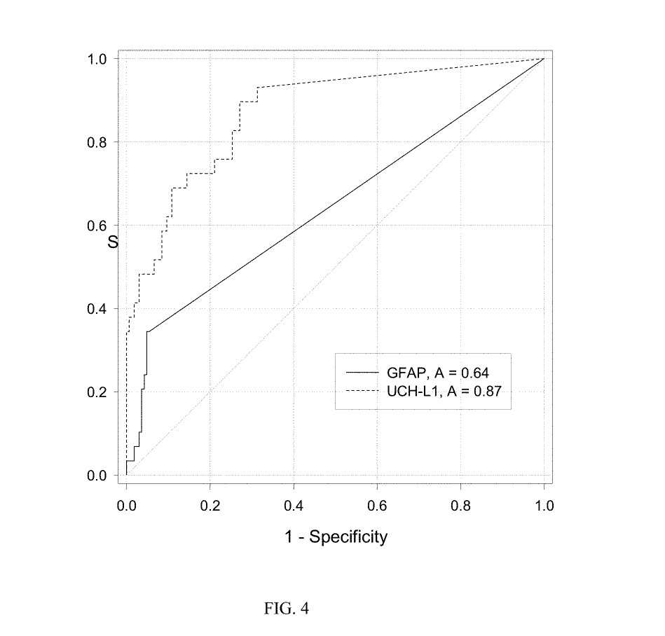

FIG. 5 illustrates the level of GFAP, UCH-L1, and S100β in serum from TBI human subjects with mild and moderate injury magnitude;

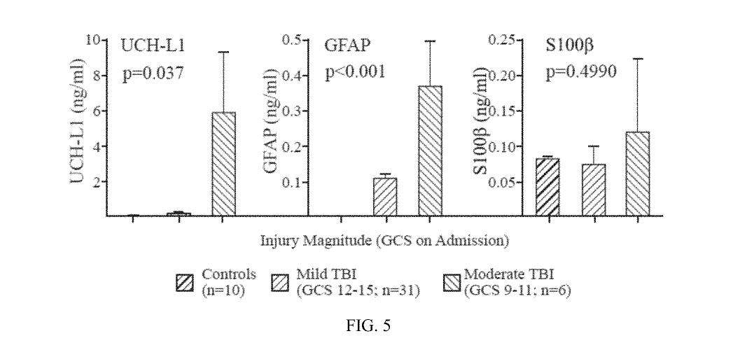

FIG. 6 is a schematic view of the in vitro diagnostic device;

FIG. 7 represents UCH-L1, GFAP, S100β, NSE, MBP, and MAP2 amounts present in serum post severe traumatic brain injury in human subjects as a function of CT scan results;

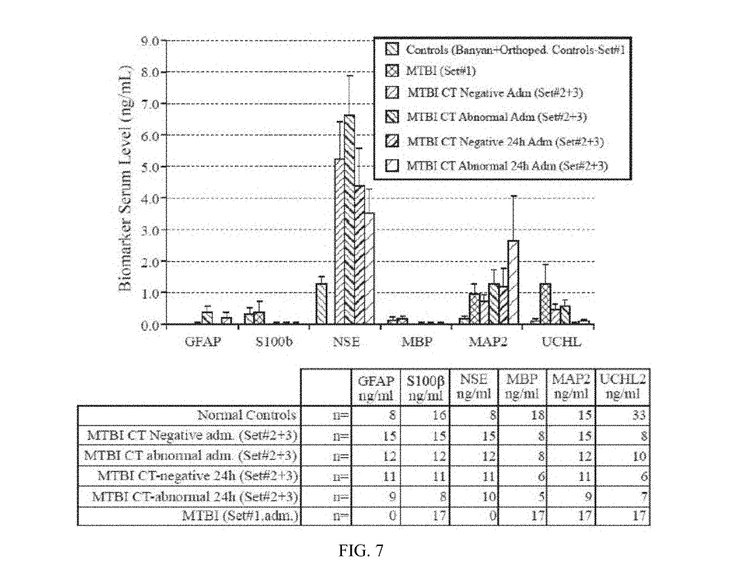

FIG. 8 illustrates a 2D gel detecting the brain specific or brain enriched isoforms of S100β in serum, showing the capture of the isoforms represented by SEQ ID NO 2-6, which represent the S100β isoforms, S10002-S10006, respectively.

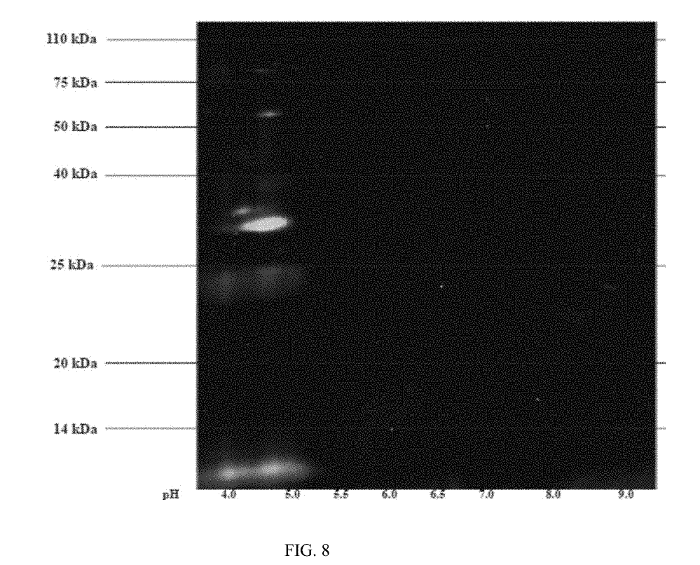

FIG. 9 illustrates a western blot tissue panel showing the brain enriched nature of S100β2.

### DETAILED DESCRIPTION OF THE PREFERRED EMBODIMENTS

The present invention has utility in the diagnosis and management of abnormal neurological condition. Through the measurement of biomarkers in a biological sample from a subject, such as the neural specific and brain enriched S100β isoforms, S100β2-S100β6, alone or in combination with each other or in combination with one or more additional neuroactive markers selected from GFAP, UCH-L1, NSE, MBP, MAP2, SBDP, CRMP, CNPase, NRP-2 synaptotagmin, or neurensin-1 (p24) from a subject, a determination of subject neurological condition is provided with greater specificity than previously attainable. The present description is directed toward a first isoform of S100β, selected from S100β1-S100β6, as a biomarker. It is appreciated that the invention encompasses several other additional biomarkers illustratively, including GFAP, UCH-L1, NSE, MAP2, and SBDP. Surprisingly, by combining the detection of more than one biomarker, a synergistic result is achieved. Illustratively, combining the detection of two neuroactive biomarkers, such as S100β2 and UCH-L1 or GFAP, provides sensitive detection that is unexpectedly able to discern the level and severity of an abnormal neurological condition in a subject. It should also be appreciated that other neural specific forms of S100β, represented by any of SEQ. ID. NO. 2-6, provide this synergistic effect when measured in combination with UCH-L1 or GFAP. An even greater synergy and assay specificity is achieved by combining a neural specific or neural enriched isoform of S100β in an assay with UCH-L1 or GFAP.

The inventive compositions of matter, methods, processes, kits, and in vitro diagnostic devices allow for a simple, yet sensitive, clinical approach to diagnosing damage to the CNS, neurotoxicity, brain injury, and neuronal disorders using biological fluid particularly measuring for one or more of a biomarker classified as a S100 calcium binding protein β (S100β) isoform, namely S100β2, S100β3, S100β4, S100β5 and S100β6. In addition, the foregoing also allows for the synergistic combination of such isoforms with other clinically relevant neural markers such as such as glial fibrillary acidic protein (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) in particular SBDP150, SBDP150i SBDP145, SBDP120, vesicular membrane protein neurensin-1 (p24), collapsin response mediated proteins (CRMP's) and breakdown products thereof, or synaptotagmin and breakdown products thereof. Inventive markers include proteins or protein fragments, autoantibodies, DNA, RNA, or miRNA.

As used herein the term “diagnosing” means recognizing the presence or absence of a neurological or other condition, such as an injury or disease. Diagnosing is in a subset of circumstances referred to as the result of an assay wherein a particular ratio or level of a biomarker is detected or is absent.

As used herein a “ratio” is either a positive ratio, wherein the level of the target is greater than the target in a second sample or relative to a known or recognized baseline level of the same target; a negative ratio, wherein the level of the target is lower than the target in a second sample or relative to a known or recognized baseline level of the same target; or a neutral ratio, wherein there is no observed change in target biomarker.

As used herein an injury is an alteration in cellular or molecular integrity, activity, level, robustness, state, or other alteration that is traceable to an event. Injury illustratively includes a physical, mechanical, chemical, biological, functional, infectious, or other modulator of cellular or molecular characteristics. An event is illustratively a physical trauma, such as an impact (percussive), or a biological abnormality, such as a stroke resulting from either blockade or leakage of a blood vessel. An event is optionally an infection by an infectious agent. A person of skill in the art recognizes numerous equivalent events that are encompassed by the terms injury or event.

An injury is in some instances a physical event, such as a percussive impact. An impact is the like of a percussive injury, such as resulting from a blow to the head that either leaves the cranial structure intact or results in breach thereof. Experimentally, several impact methods are used illustratively including controlled cortical impact (CCI) at a 1.6 mm depression depth, equivalent to severe TBI in humans. This method is described in detail by Cox, C D, et al., J Neurotrauma, 2008; 25(11):1355-65. It is appreciated that other experimental methods producing impact trauma are similarly operable.

As used herein Coma shall mean the initial stage after a severe brain injury, a state of unconsciousness. People in a coma are unaware and unresponsive, but not asleep as there is no sleep-wake cycle. While in a coma, people are unable to speak, follow commands or open their eyes. As a person's GCS score improves, he or she is considered to be emerging from the coma. These changes usually take place gradually. For instance, eyes may open or there may be evidence of sleep cycles, but still no ability to speak or follow commands. As these abilities appear, most rehabilitation centers will use the Rancho Los Amigos Cognitive Scale (see separate document) to describe progress after this point.

As used herein “vegetative state” shall mean the period where emergence from coma can seem to stop before the person becomes conscious. People in a vegetative state may open their eyes and have sleep-wake cycles, but are still unconscious. Although not considered to be in a coma, the patients remain totally unaware. In a vegetative state, any apparent signs of responding to surroundings are reflexes and not indications of awareness. The term “permanent vegetative state” is used only when a person is determined to be in a vegetative state for twelve months after trauma or three months after a brain injury that caused oxygen insufficiency. Always discuss with the physician questions about responses and awareness.

As used herein “minimally conscious state” refers to people who demonstrate some, but very little, awareness and responsiveness to their surroundings. Responses are typically inconsistent and thus neither considered comatose nor vegetative. As the name suggests, a person is considered conscious in this state. Occasionally, physicians may prescribe medicines that help stimulate the brain, especially if a person is not becoming more responsive with time. Some people do not progress beyond this stage in their recovery process. Ischemic stroke is readily modeled by middle cerebral artery occlusion (MCAO) in rodents. UCH-L1 protein levels, for example, are increased following mild MCAO which is further increased following severe MCAO challenge. Mild MCAO challenge may result in an increase of protein levels within two hours that is transient and returns to control levels within 24 hours. In contrast, severe MCAO challenge results in an increase in protein levels within two hours following injury and may be much more persistent demonstrating statistically significant levels out to 72 hours or more.

The present invention provides for the detection of a neurological condition in a subject. A neurological condition may be an abnormal neurological condition such as that caused by genetic disorder, injury, or disease to nervous tissue. As such, it is a further object of the present invention to provide a method for detecting or diagnosing an abnormal neurological condition in a subject.

It is to be understood that in instances where a range of values is provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-3, 2-4, 3-4, and 1-4.

The present invention also provides an assay for detecting or diagnosing the neurological condition of a subject. As the neurological condition may be the result of stress, such as that from exposure to environmental, therapeutic, or investigative compounds, it is a further aspect of the present invention to provide a process and assay for screening candidate drug or other compounds or for detecting the effects of environmental contaminants regardless of whether the subject itself or cells derived there from are exposed to the drug candidate or other possible stressors.

For purposes of the subject invention, brain injury is divided into two levels, mild traumatic brain injury (MTBI), and traumatic brain injury (TBI). An intermediate level of moderate TBI is also referred to herein. The spectrum between MTBI and extending through moderate TBI is also referred to synonymously as mild to moderate TBI or by the abbreviation MMTBI.

### In Vitro Diagnostic Device

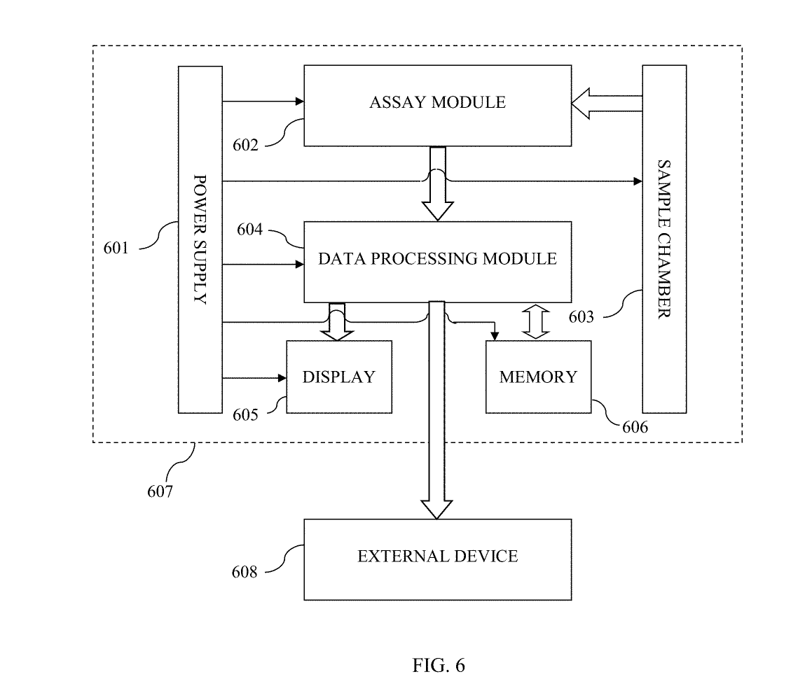

FIG. 6 schematically illustrates an inventive in vitro diagnostic device. An inventive in vitro diagnostic device includes of at least a sample collection chamber 603, an assay module 602 used to detect biomarkers of neural injury or neuronal disorder, and a user interface that relates the amount of the measured biomarker measured in the assay module. The in vitro diagnostic device is readily fashioned in the form of a handheld device, a bench top device, or a point of care device.

The sample chamber 603 can be of any sample collection apparatus known in the art for holding a biological fluid. In one embodiment, the sample collection chamber can accommodate any one of the biological fluids herein contemplated, such as whole blood, plasma, serum, urine, sweat, or saliva.

The assay module 602 includes any type of an assay which may be used for detecting a protein antigen in a biological sample, for instance, through the use of antibodies in an immunoassay. The assay module 602 includes any variety of an assay currently known in the art; however the assay should be optimized for the detection of neural biomarkers used for detecting neural injury or neuronal disorder in a subject. The assay module 602 is in fluid communication with the sample collection chamber 603. In one embodiment, the assay module 602 includes an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. In one embodiment, a colorimetric assay may be used which includes only a sample collection chamber 603 and an assay module 602 of the assay. Although not specifically shown, these components are in some inventive embodiments housed in one assembly 607. In one embodiment the assay module 602 contains an agent specific for detecting one or more of the biomarkers of glial fibrillary acidic protein (GFAP) or one or more GFAP breakdown products that correlates with a quantity of whole GFAP, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) that in particular inventive embodiments are SBDP150, SBDP150i SBDP145, SBDP120, S100 calcium binding protein B (S100b), collapsin response mediated proteins (CRMP's) or one or more breakdown products that correlates with a quantity of whole CRMP, or synaptotagmin or one or more breakdown products that correlates with a quantity of whole synaptotagmin. The assay module 602 may contain additional agents to detect additional biomarkers, as is described herein.

In one embodiment, the inventive in vitro diagnostic device contains a power supply 601, an assay module 602, a sample chamber 603, and a data processing module 605. The power supply 601 is electrically connected to the assay module and the data processing module. The assay module 602 and the data processing module 605 are in electrical communication with each other. As described above, the assay module 602 is any assay currently known in the art; however the assay should be optimized for the detection of neural biomarkers used for detecting neural injury or neuronal disorder in a subject. The assay module 602 is in fluid communication with the sample collection chamber 603. The assay module 602 includes an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoas say, chemiluminescent immunoas say, phosphorescent immunoassay, or an anodic stripping voltammetry immunoassay. A biological sample is placed in the sample chamber 603 and assayed by the assay module 602 detecting for a biomarker of neural injury or neuronal disorder. The measured amount of the biomarker by the assay module 602 is then electrically communicated to the data processing module 604. The data processing 604 module includes any known data processing element known in the art, and can include a microelectronic chip, a central processing unit (CPU), or a software package which processes the information supplied from the assay module 602.

In one embodiment, the data processing module 604 is in electrical communication with a display 605, a memory device 606, or an external device 608 or software package (such as laboratory and information management software (LIMS)). In one embodiment, the data processing module 604 is used to process the data into a user defined usable format. This format includes the measured amount of neural biomarkers detected in the sample, indication that a neural injury or neuronal disorder is present, or indication of the severity of the neural injury or neuronal disorder. The information from the data processing module 604 may be illustrated on the display 605, saved in machine readable format to a memory device, or electrically communicated to an external device 608 for additional processing or display. Although not specifically shown, these components are in some inventive embodiments housed in one assembly 607. In one embodiment, the data processing module 604 may be programmed to compare the detected amount of the biomarker transmitted from the assay module 602, to a comparator algorithm. The comparator algorithm may compare the measured amount to the user defined threshold which may be any limit useful to the user. In one embodiment, the user defined threshold is set to the amount of the biomarker measured in control subject, or a statistically significant average of a control population.

In one embodiment, an in vitro diagnostic device includes one or more tools, or equipment configured to hold or collect a biological sample from an individual. In one embodiment of an in vitro diagnostic device, tools to collect a biological sample may include one or more of a swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. These reagents include those peptides represented by SEQ. ID No. 1-6, or antibodies raised against said SEQ ID NO. Such reagents and solutions for nucleotide collecting, stabilizing, storing, and processing are well known by those of skill in the art and may be indicated by specific methods used by an in vitro diagnostic test as described herein. In one embodiment, an in vitro diagnostic device, as disclosed herein, includes a micro array apparatus and reagents, a flow cell apparatus and reagents, a multiplex nucleotide sequencer and reagents, and additional hardware and software necessary to assay a genetic sample for certain genetic markers and to detect and visualize certain biological markers.

### Kits

In yet another aspect, the invention provides kits for aiding a diagnosis of neural injury, degree of severity of injury, subcellular localization, and/or neuronal disorders, wherein the kits can be used to detect the markers of the present invention. For example, the kits can be used to detect any one or more of the markers described herein, including which markers are differentially present in samples of a patient and normal subjects. The kits of the invention have many applications. For example, the kits can be used to differentiate if a subject has axonal injury versus, for example, dendritic, or has a negative diagnosis, thus aiding neuronal injury diagnosis. In another example, the kits can be used to identify compounds that modulate expression of one or more of the markers in in vitro or in vivo animal models to determine the effects of treatment.

In one embodiment, a kit includes (a) an antibody that specifically binds to a marker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further include pre-fractionation spin columns. In one embodiment, the kit may further include instructions for suitable operation parameters in the form of a label or a separate insert.

In another embodiment, the kit includes (a) a panel or composition of detecting agent to detect a panel or composition of biomarkers. The panel or composition of reagents included in a kit provide for the ability to detect at least one biomarker and/or a plurality of biomarkers in order to diagnose a neural injury. These biomarkers shall first detect for at least one neural specific or brain specific or brain enriched isoform of S100β, namely S100β2, S100β3, S100β4, S100β5, or S100β6. The kit may additionally include reagents to test for other biomarkers such as GFAP, ubiquitin carboxyl-terminal esterase L1 (UCH-L1), Neuron specific enolase (NSE), spectrin breakdown products (SBDP), that in particular inventive embodiments are SBDP150, SBDP150i SBDP145, SBDP120, S100 β1, microtubule associated proteins (MAP), that in particular inventive embodiments are MAP2, MAP1, MAP3, MAP4, MAP5, myelin basic protein (MBP), Tau, Neurofilament protein (NF), Cannabinoid Receptor (CB), CAM proteins, Synaptic protein, collapsin response mediator proteins (CRMP), inducible nitric oxide synthase (iNOS), Neuronal Nuclei protein (NeuN), 2′,3′-cyclic nucleotide-3′-phosphohydrolase (CNPase), Neuroserpin, alpha-internexin, microtubule-associated protein 1 light chain 3 (LC3), Neurofascin, the glutamate transporters (EAAT), Nestin, Cortin-1, 2′, and BIII-Tubulin.

In one embodiment, the invention includes a diagnostic kit for use in screening serum containing antigens of the peptide of the invention. These antigens may be one of any of the peptides represented by SEQ ID NO. 1-6. The diagnostic kit includes a substantially isolated antibody specifically immunoreactive with peptide or polynucleotide antigens, and a method detecting the binding of the polynucleotide or peptide antigen to the antibody. In one embodiment, the antibody is attached to a solid support. Antibodies used in the inventive kit are those raised against any one of the peptides represented in SEQ ID NO's 1-6. In one embodiment, the antibody is a monoclonal or polyclonal antibody raised against the rat, rabbit, or human forms of the brain or neural specific isoforms of S100β described herein. The detecting means of the kit includes a second, labeled monoclonal or polyclonal antibody. Alternatively, or in addition thereto, the detecting means includes a labeled, competing antigen.

In one diagnostic configuration, test serum is reacted with a solid phase reagent having a surface-bound antigen obtained by the methods of the present invention. After binding the specific antigen and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate, or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Optionally, the kit may further include a standard or control information so that the test sample can be compared with the control information standard to determine if the test amount of a marker detected in a sample is a diagnostic amount consistent with a diagnosis of neural injury, degree of severity of the injury, subcellular localization, neuronal disorder, and/or effect of treatment on the patient.

In one embodiment, a kit includes: (a) a substrate comprising an adsorbent thereon, wherein the adsorbent is suitable for binding a marker; and (b) instructions to detect the marker or markers by contacting a sample with the adsorbent and detecting the marker or markers retained by the adsorbent. In some embodiments, the kit may include an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In one embodiment, the kit includes a first substrate comprising an adsorbent thereon (e.g., a particle functionalized with an adsorbent) and a second substrate onto which the first substrate can be positioned to form a probe which is removably insertable into a gas phase ion spectrometer. Alternatively, the kit may include a single substrate which is in the form of a removably insertable probe with adsorbents on the substrate. The kit may further include a pre-fractionation spin column (e.g., Cibacron blue agarose column, anti-HSA agarose column, size exclusion column, Q-anion exchange spin column, single stranded DNA column, lectin column, etc.).

Optionally, the kit can further include instructions for suitable operational parameters in the form of a label or a separate insert. For example, the kit may have standard instructions informing a consumer about how to wash the probe after a sample is contacted on the probe. In another example, the kit may have instructions for pre-fractionating a sample to reduce complexity of proteins in the sample. In another example, the kit may have instructions for automating the fractionation or other processes.

### Biological Fluids

The inventive compositions of matter, processes, methods, and in vitro diagnostic devices provide the ability to detect and monitor levels of those neural proteins or autoantibodies which are released into the body after neurotoxicity or CNS injury to provide enhanced diagnostic capability by allowing clinicians (1) to determine the level of injury severity in patients with various CNS injuries; (2) to monitor patients for signs of secondary CNS injuries that may elicit these cellular changes; and (3) to continually monitor the progress of the injury and the effects of therapy by examination of these proteins in biological fluids, such as blood, plasma, serum, CSF, urine, saliva, or sweat. Unlike other organ-based diseases where rapid diagnostics for surrogate biomarkers prove invaluable to the course of action taken to treat the disease, no such rapid, definitive diagnostic tests exist for traumatic or ischemic brain injury that might provide physicians with quantifiable neurochemical markers to help determine the seriousness of the injury, the anatomical and cellular pathology of the injury, and the implementation of appropriate medical management and treatment.

A sample is in some inventive embodiments a biological sample. Particular examples of biological samples are illustratively cells, tissues, cerebral spinal fluid (CSF), artificial CSF, whole blood, serum, plasma, cytosolic fluid, urine, feces, stomach fluids, digestive fluids, saliva, nasal or other airway fluid, vaginal fluids, semen, buffered saline, saline, water, or other biological fluid recognized in the art. Preferably, the biological samples include CSF, blood, serum, plasma, sweat, saliva, and urine. It should be appreciated that after injury to the nervous system (such as brain injury), the neural cell membrane is compromised, leading to the efflux of neural proteins first into the extracellular fluid or space and also to the cerebrospinal fluid. Eventually the neural proteins efflux to the circulating blood (as assisted by the compromised blood brain barrier) and, through normal bodily function (such as impurity removal from the kidneys), the neural proteins migrate to other biological fluids such as urine, sweat, and saliva. Thus, other suitable biological samples include, but are not limited to, such cells or fluid secreted from these cells. It should also be appreciated that obtaining biological fluids, such as cerebrospinal fluid, blood, plasma, serum, saliva, and urine, from a subject is typically much less invasive and traumatizing than obtaining a solid tissue biopsy sample. Thus, samples, which are biological fluids, are used in particular embodiments of the invention.

Biological samples of CSF, blood, urine, and saliva are collected using normal collection techniques. For example, and not to limit the sample collection to the procedures contained herein, in CSF Lumbar Puncture (LP) a 20-gauge introducer needle is inserted and an amount of CSF is withdrawn. For blood, the samples may be collected by venipuncture in Vacutainer tubes and spun down and separated into serum and plasma in particular embodiments of the invention. For urine and saliva, samples are collected, preferably avoiding the introduction of contaminants into the specimen. All biological samples may be stored in aliquots at 80° C. for later assay. Surgical techniques for obtaining solid tissue samples are well known in the art. For example, methods for obtaining a nervous system tissue sample are described in standard neurosurgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in Belay et al., Arch. Neurol. 58: 1673-1678 (2001); and Seijo et al., J. Clin. Microbiol. 38: 3892-3895 (2000). Any suitable biological samples can be obtained from a subject to detect markers. It should be appreciated that the methods employed herein may be identically reproduced for any biological fluid to detect a marker or markers in a sample.

After insult, the damaged tissue, organs, or nerve cells in in vitro culture or in situ in a subject express altered levels or activities of one or more proteins than do such cells not subjected to the insult. Thus, samples that contain nerve cells, e.g., a biopsy of a CNS or PNS tissue are illustratively suitable biological samples for use in the invention.

A subject illustratively includes a dog, a cat, a horse, a cow, a pig, a sheep, a goat, a chicken, a non-human primate, a human, a rat, and a mouse. Subjects who most benefit from the present invention are those suspected of having or at risk for developing abnormal neurological conditions, such as victims of brain injury caused by traumatic insults (e.g., gunshot wounds, automobile accidents, sports accidents, shaken baby syndrome), ischemic events (e.g., stroke, cerebral hemorrhage, cardiac arrest), neurodegenerative disorders (e.g., Alzheimer's, Huntington's, and Parkinson's diseases; prion-related disease; other forms of dementia), epilepsy, substance abuse (e.g., from amphetamines, Ecstasy/MDMA, or ethanol), and PNS pathologies such as diabetic neuropathy, chemotherapy-induced neuropathy, and neuropathic pain.

Baseline levels of several biomarkers are those levels obtained in the target biological sample in the species of desired subject in the absence of a known neurological condition. These levels need not be expressed in hard concentrations, but may instead be known from parallel control experiments and expressed in terms of fluorescent units, density units, and the like. Typically, baselines are determined from subjects where there is an absence of a biomarker or present in biological samples at a negligible amount. However, some proteins may be expressed less in an injured patient. Determining the baseline levels of protein biomarkers in a particular species is well within the skill of the art. Similarly, determining the concentration of baseline levels of neural or liver injury biomarkers is well within the skill of the art.

### Neural Biomarkers

The inventive processes, methods, compositions of matter, and devices in some inventive embodiments include determining the neurological condition of a subject by assaying a sample derived from a subject at a first time for the presence of at least one biomarker wherein said at least one biomarker is a neural specific or brain specific or brain enriched isoform of S100β, namely S100β2, S100β3, S100β4, S100β5, and S100β6. A biomarker is a cell, protein, nucleic acid, steroid, fatty acid, metabolite, or other differentiator useful for measurement of biological activity or response. Additional biomarkers operable herein illustratively include: ubiquitin carboxyl-terminal esterase, ubiquitin carboxy-terminal hydrolase, spectrin breakdown product(s), a neuronally-localized intracellular protein, MAP-tau, C-tau, MAP2, poly (ADP-ribose) polymerase (PARP), collapsin response mediator protein, Annexin All, Aldehyde dehydrogenase family 7, Cofilin 1, Profilin 1, α-Enolase (non-neural enolase), Enolase 1 protein, Glyceraldehyde-3-phosphate dehydrogenase, Hexokinase 1, Aconitase 2, Acetyl-CoA synthetase 2, Neuronal protein 22, Phosphoglycerate kinase 2, Phosphoglycerate kinase 1, Hsc70-ps 1, Glutamate dehydrogenase 1, Aldolase A, Aldolase C, fructose-biphosphate, Dimethylarginine dimethylaminohydrolase 1, Microtubule-associated protein 2, Carbonic anhydrase, ADP-ribosylation factor 3, Transferrin, Liver regeneration-related protein, Hemoglobin α-chain, Hemoglobin β chain, Liver regeneration-related protein, Fetuin β, 3-Oxoacid-CoA transferase, Malate dehydrogenase 1, NAD (soluble), Lactate dehydrogenase B, Malate dehydrogenase, Carboxylesterase E1 precursor, Serine protease inhibitor al, Haptoglobin, Ubiquitin carboxyl-terminal hydrolase L1, Serine protease inhibitor 2a, T-kininogen, al major acute phase protein, Albumin, al major acute phase protein prepeptide, Murinoglobulin 1 homolog, Group-specific component protein, Guanosine diphosphate dissociation inhibitor 1, Collapsin response mediator protein 2, Murinoglobulin 1 homolog, Ferroxidase, Ceruloplasmin, Spectrin α-chain, brain, C-reactive protein, Brain creatine kinase, Proteasome subunit α-type 7, 14-3-3 protein, Synaptotagmin, subtypes thereof, fragments thereof, breakdown products thereof, or combinations thereof. Other potential biomarkers illustratively include those identified by Kobeissy, F H, et al, Molecular & Cellular Proteomics, 2006; 5:1887-1898, the contents of which are incorporated herein by reference, or others known in the art. However it should be appreciated that S100β2, S100β3, S100β4, S100β5, and S100β6 may be used in an assay alone, in combination with each other, or alternatively combined or multiplexed with other markers, that in particular inventive embodiments are UCH-L1, GFAP, spectrin breakdown products (SBDP), that in particular inventive embodiments are SBDP150, SBDP150i SBDP145, SBDP120, S100β1, CRMP's, Synaptotagmin, Cannabinoid Receptor (CB), MAP-2, or neurofilament proteins (NF-H. NF-M, NF-L).

It is appreciated, however, that multiple biomarkers may be predictors of different modes or types of damage to the same cell type. Through the use of an inventive assay inclusive of biomarkers associated with glial cells, as well as at least one other type of neural cell, the type of neural cells being stressed or killed, as well as quantification of neurological condition results, provides rapid and robust diagnosis of traumatic brain injury type. Measuring a neural or brain specific or brain enriched isoform of S100β along with at least one additional neuroactive biomarker, and comparing the quantity of the S100β isoform and the additional biomarker to normal levels of the markers, provides a determination of subject neurological condition.

In some inventive embodiments, specific biomarker levels that, when measured in concert with S100β isoforms, afford superior evaluation of subject neurological condition include GFAP, SBDP 150, SBDP150i, a combination of SBDP145 (calpain mediated acute neural necrosis) and SBDP120 (caspase mediated delayed neural apoptosis), UCH-L1 (neuronal cell body damage marker), and MAP2. This is noted to be of particular value in measuring MMTBI, predicting abnormal CT scans, determining neurotoxicity, distinguishing stroke from TBI, or subarachnoid hemorrhage.

To provide correlations between a neurological condition and measured quantities of a neural specific isoform of S100β alone or in combination with each other or other neuroactive biomarkers, biological samples are collected from subjects with the samples being subjected to measurement for these biomarkers. The subjects vary in neurological condition. Detected levels of a neural specific isoform of S100β alone or in combination with each other or other neuroactive biomarkers are optionally then correlated with CT scan results, as well as GCS scoring. Based on these results, an inventive assay is developed and validated (Lee et al., Pharmacological Research 23:312-328, 2006). It is appreciated that the aforementioned biomarkers, in addition to being obtained from CSF and serum, are also readily obtained from blood, plasma, saliva, urine, sweat, buccal swab, as well as solid tissue biopsy.

### Neurotoxicity Markers

In a particular embodiment, the described compositions of matter, methods, processes, kits, and in vitro diagnostic devices provide the ability to detect and monitor levels of proteins detecting a neurotoxic insult. Several biomarkers are used, for each of which an assay is developed and incorporated for use with the described methods, processes, kits, and in vitro diagnostic devices, or incorporated to use the described compositions of matter. Biomarkers of neurotoxicity include neural specific isoforms of S100β (S100β2, S100β3, S100β4, S100β5, or S100β6); ubiquitin carboxyl-terminal hydrolase-L1 (UCH-L1); spectrin; a spectrin breakdown product (SBDP); MAP1; MAP2; GFAP; ubiquitin carboxyl-terminal esterase; ubiquitin carboxyl-terminal hydrolase; a neuronally-localized intracellular protein; MAP-tau; C-tau; Poly (ADP-ribose) polymerase (PARP); a collapsin response mediator protein; synaptotagmin; βIII-tubulin; neuron-specific enolase; neurofilament protein light chain; nestin; α-internexin; breakdown products thereof; post-translationally modified forms thereof; derivatives thereof; and combinations thereof.

### Immunoassays

The detection methods and process may be implemented into assays or into kits for performing assays. These kits or assays may alternatively be packaged into a cartridge to be used with an inventive in vitro diagnostic device. Such a device makes use of these cartridges, kits, or an assay in an assay module 602, which may be one of many types of assays. The biomarkers of the invention can be detected in a sample by any means. Methods for detecting the biomarkers are described in detail in the materials and methods and Examples which follow. For example, immunoassays include but are not limited to competitive and non-competitive assay systems using techniques such as western blots, radioimmunoassays, ELISA (enzyme linked immunosorbent assay), “sandwich” immunoassays, magnetic immunoassays, radioisotope immunoassays, fluorescent immunoassays, immunoprecipitation assays, precipitin reactions, gel diffusion precipitin reactions, immunodiffusion assays, fluorescent immunoassays, chemiluminescent immunoassays, phosphorescent immunoassays, anodic stripping voltammetry immunoassays, and the like. Inventive in vitro diagnostic devices may also include any known devices currently available that utilize ion-selective electrode potentiometry, microfluids technology, fluorescence or chemiluminescence, or reflection technology that optically interprets color changes on a protein test strip. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation). It should be appreciated that, at present, none of the existing technologies present a method of detecting or measuring any of the ailments disclosed herein, nor does there exist any method of using such in vitro diagnostic devices to detect any of the disclosed biomarkers to detect their associated injuries.

### Compositions of Matter

An exemplary process for detecting the presence or absence of a biomarker, alone or in combination, in a biological sample involves obtaining a biological sample from a subject, such as a human; contacting the biological sample with a compound or an agent capable of detecting of the marker being analyzed; illustratively including an antibody or aptamer; and analyzing binding of the compound or agent to the sample after washing. Those samples having specifically bound compound or agent express the marker being analyzed.

For example, in vitro techniques for detection of a marker illustratively include enzyme linked immunosorbent assays (ELISAs), radioimmunoassay, radioassay, western blot, Southern blot, northern blot, immunoprecipitation, immunofluorescence, mass spectrometry, RT-PCR, PCR, liquid chromatography, high performance liquid chromatography, enzyme activity assay, cellular assay, positron emission tomography, mass spectroscopy, combinations thereof, or other technique known in the art. Furthermore, in vivo techniques for detection of a marker include introducing a labeled agent that specifically binds the marker into a biological sample or test subject. For example, the agent can be labeled with a radioactive marker whose presence and location in a biological sample or test subject can be detected by standard imaging techniques. Optionally, the first biomarker specifically binding agent and the agent specifically binding at least one additional neuroactive biomarker are both bound to a substrate. It is appreciated that a bound agent assay is readily formed with the agents bound with spatial overlap, with detection occurring through discernibly different detection for first biomarker and each of at least one additional neuroactive biomarkers. A color intensity based quantification of each of the spatially overlapping bound biomarkers is representative of such techniques.

Any suitable molecule that can specifically bind to a biomarker and any suitable molecule that specifically binds one or more other biomarkers of a particular condition are operative in the invention to achieve a synergistic assay. An agent for detecting the one or more biomarkers of a condition according to the present invention is an antibody capable of binding to the biomarker being analyzed. Preferably, an antibody is conjugated with a detectable label. Such antibodies can be polyclonal or monoclonal. An intact antibody, a fragment thereof (e.g., Fab or F(ab′)₂), or an engineered variant thereof (e.g., sFv), can also be used. Such antibodies can be of any immunoglobulin class including IgG, IgM, IgE, IgA, IgD, and any subclass thereof. Antibodies for numerous inventive biomarkers are available from vendors known to one of skill in the art. Illustratively, antibodies directed to inventive biomarkers are available from Santa Cruz Biotechnology (Santa Cruz, Calif.). Exemplary antibodies operative herein are used to detect a biomarker of the disclosed conditions. In addition, antigens to detect autoantibodies may also be used to detect chronic injury of the stated injuries and disorders.

An antibody is optionally labeled. A person of ordinary skill in the art recognizes numerous labels operable herein. Labels and labeling kits are commercially available optionally from Invitrogen Corp (Carlsbad, Calif.). Labels illustratively include fluorescent labels, biotin, peroxidase, radionucleotides, or other label known in the art. Alternatively, a detection species of another antibody or other compound known to the art is used as form detection of a biomarker bound by an antibody.

Antibody-based assays are used in particular embodiments of the present invention for analyzing a biological sample for the presence of a biomarker and one or more other biomarkers of a particular injury or condition. Suitable western blotting methods are described below in the Examples section. For more rapid analysis (as may be important in emergency medical situations), immunosorbent assays (e.g., ELISA and RIA) and immunoprecipitation assays may be used. As one example, the biological sample or a portion thereof is immobilized on a substrate, such as a membrane made of nitrocellulose or PVDF, or a rigid substrate made of polystyrene or other plastic polymer such as a microtiter plate, and the substrate is contacted with an antibody that specifically binds GFAP, or one of the other neuroactive biomarkers under conditions that allow binding of antibody to the biomarker being analyzed. After washing, the presence of the antibody on the substrate indicates that the sample contained the marker being assessed. If the antibody is directly conjugated with a detectable label, such as an enzyme, fluorophore, or radioisotope, the presence of the label is optionally detected by examining the substrate for the detectable label. Alternatively, a detectably labeled secondary antibody that binds the marker-specific antibody is added to the substrate. The presence of detectable label on the substrate after washing indicates that the sample contained the marker.

Reagents as described herein may be any antibody to a protein or peptide sequence or any antigen to detect an antibody formed as part of an autoimmune response in a subject. In particular for the antigens used for these detection methods and devices, they may be those proteins or peptides generated to detect an antibody that has been produced by a subject's own tissues as an autoimmune response to cells, tissues, or native proteins of the organism in which it was formed. These antigens in some inventive embodiments are peptides having the following sequence for each of the defined isoforms from which antibodies are, have been, or will be generated:

SEQ ID NO. 1 is the publicly recognized (LOCUS NP_006263 92; CAG46920, aa (molecular wt. about 10,713 Daltons) linear, DEFINITION protein S100β [Homo sapiens], ACCESSION NP_006263 VERSION NP_006263.1 GI: 5454034 or GI: 4957424 DBSOURCE REFSEQ: accession NM_006272.2; embl accession CR542123.1) generic sequence commonly referred to or linked to all S100β studies currently recognized in the art. Because of the recent discoveries of the several isoforms, not previously known in the art, the conventional name S100β necessarily needs to be changed to S100β1 as is the convention within the scientific research community. This peptide, or antibodies raised against this peptide, is recognized in the art as not being neural specific. This protein is known to have the following sequence, as noted is the result of splicing exon 1 and exon 2:

MSELEKAMVALIDVFHQYSGREGDKHKLKKSELKELINNELSHFLEEIKE
QEVVDKVMETLDNDGDGECDFQEFMAFVAMVTTACHEFFEHE

SEQ ID NO 2 is the first of the several newly discovered compositions of matter formed as a result of the splicing exon 1, exon 2 (truncated #1), and the newly discovered exon 3 of the human peptide sequence, thus named S100β2. The neural specific peptide S100β2, found to have a molecular weight of an estimated 11,295 Daltons, has been found to have the following sequence:

MSELEKAMVALIDVFHQYSGREGDKHKLKKSELKELINNELSHFLEEIKE
QEVVDKVMETLDNDGDGECDFQEFMAFVAMRRSCKKADSKGLQPSRS

SEQ ID NO 3 is the second of the several newly discovered compositions of matter formed as a result of exon 1 (truncated) and exon 3 of the human peptide sequence, thus named S100β3. The neural specific peptide S100β3, found to have a molecular weight of about 2,750 Daltons, has been found to have the following sequence: MSELEKAMRRSCKKADSKGLQPSRS

SEQ ID NO 4 is the third of the several newly discovered compositions of matter formed as a result of exon 1 (truncated) and exon 2 of the human peptide sequence, thus named S100β4. The neural specific peptide S100β4, found to have a molecular weight of about 5,950 Daltons, has been found to have the following sequence:

MSELEKAMEIKEQEVVDKVMETLDNDGDGECDFQEFMAFVAMVTTACHEF
FEHE

SEQ ID NO 5 is the fourth of the several newly discovered compositions of matter formed as a result of exon 1 (truncated), exon 2 (truncated #1), and exon 3 of the human peptide sequence, thus named S100β5. The neural specific peptide S100β5, found to have a molecular weight of about 6,500 Daltons, has been found to have the following sequence:

MSELEKAMEIKEQEVVDKVMETLDNDGDGECDFQEFMAFVAMRRSCKKADS
KGLQPSRS

SEQ ID NO 6: is the fifth of the several newly discovered compositions of matter formed as a result of exon 1, exon 2 (truncated #2), and exon 3 of the human peptide sequence, thus named S100β6. The neural specific peptide S100β6, found to have a molecular weight of about 7,600 Daltons, has been found to have the following sequence:

MSELEKAMVALIDVFHQYSGREGDKHKLKKSELKELINNELSHFLEEIKEQ
ERRSCKKADSKGLQPSRS

It should be appreciated that, while the sequences described above are those S100β isoforms found for the human peptide sequence, similar isoforms are available in rat, mouse, rabbit, bovine, Chinese hamster, Rhesus monkey, zebrafish, goat, chicken, dog, domestic cat, pig, and other species having a similar sequence alignment to the S100β for humans. These alternative sequences may be substituted in the inventive processes, methods, assays, and in vitro diagnostic devices described herein. In addition, while discovery of these proteins are a landmark discovery in diagnostics of neural injuries, other compositions of matter of importance are those antibodies raised against any of the peptide sequences represented by SEQ ID NO's 1-6, or antibodies raised against the rat, mouse, rabbit, bovine, Chinese hamster, Rhesus monkey, zebrafish, goat, chicken, dog, domestic cat, pig, and other similar species formulations of the similar sequence alignment.

Numerous permutations of these basic immunoassays are also operative in the invention. These include the biomarker-specific antibody, as opposed to the sample being immobilized on a substrate, and the substrate making contact with a biomarker conjugated with a detectable label under conditions that cause binding of antibody to the labeled marker. The substrate is then contacted with a sample under conditions that allow binding of the marker being analyzed to the antibody. A reduction in the amount of detectable label on the substrate after washing indicates that the sample contained the marker.

In other embodiments of the present invention, other suitable agents for isoform detection include a molecule (e.g., a peptide, an aptamer, or a small organic molecule) that specifically binds a biomarker being optionally used in place of the antibody in the above described immunoassays. For example, an aptamer that specifically binds all spectrin and/or one or more of its SBDPs might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

A myriad of detectable labels that are operative in a diagnostic assay for biomarker expression is known in the art. Agents used in methods for detecting a biomarker are conjugated to a detectable label, e.g., an enzyme, such as horseradish peroxidase. Agents labeled with horseradish peroxidase can be detected by adding an appropriate substrate that produces a color change in the presence of horseradish peroxidase. Several other detectable labels that may be used are known. Common examples of these include alkaline phosphatase, horseradish peroxidase, fluorescent compounds, luminescent compounds, colloidal gold, magnetic particles, biotin, radioisotopes, and other enzymes. It is appreciated that a primary/secondary antibody system is optionally used to detect one or more biomarkers. A primary antibody that specifically recognizes one or more biomarkers is exposed to a biological sample that may contain the biomarker of interest. A secondary antibody with an appropriate label that recognizes the species or isotype of the primary antibody is then contacted with the sample such that specific detection of the one or more biomarkers in the sample is achieved.

The present invention provides a step of comparing the quantity of one or more biomarkers to normal levels to determine the neurological condition of the subject. It is appreciated that selection of additional biomarkers allows one to identify the types of cells implicated in an abnormal organ or physical condition, as well as the nature of cell death in the case of an axonal injury marker, namely an SBDP. The practice of an inventive process provides a test which can help a physician determine suitable therapeutics to administer for optimal benefit of the subject. While the neural data provided in the examples herein are provided with respect to a full spectrum of traumatic brain injury, neurotoxicity, and neuronal cell death, it is appreciated that these results are applicable to other ischemic events, neurodegenerative disorders, prion related disease, epilepsy, chemical etiology, and PNS pathologies. As is shown in the subsequently provided example data, a gender difference is unexpectedly noted in abnormal subject neurological condition.

The results of such a test using an in vitro diagnostic device can help a physician determine whether the administration a particular therapeutic or treatment regimen may be effective, and provide a rapid clinical intervention to the injury or disorder to enhance a patient's recovery.

It is appreciated that other reagents, such as assay grade water, buffering agents, membranes, assay plates, secondary antibodies, salts, and other ancillary reagents, are available from vendors known to those of skill in the art. Illustratively, assay plates are available from Corning, Inc. (Corning, N.Y.) and reagents are available from Sigma-Aldrich Co. (St. Louis, Mo.).

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

### EXAMPLES

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian tissue, specifically, analyses of mouse tissue, a person having ordinary skill in the art recognizes that similar techniques and other techniques known in the art readily translate the examples to other mammals such as humans. Reagents illustrated herein are commonly cross reactive between mammalian species or alternative reagents with similar properties are commercially available, and a person of ordinary skill in the art readily understands where such reagents may be obtained. Variations within the concepts of the invention are apparent to those skilled in the art.

### Example 1: Materials for Biomarker Analyses

Illustrative reagents used in performing the subject invention include Sodium bicarbonate (Sigma Cat #: C-3041), blocking buffer (Starting block T20-TBS) (Pierce Cat#: 37543), Tris buffered saline with Tween 20 (TBST; Sigma Cat #: T-9039). Phosphate buffered saline (PBS; Sigma Cat #: P-3813); Tween 20 (Sigma Cat #: P5927); Ultra TMB ELISA (Pierce Cat #: 34028); and Nunc maxisorp ELISA plates (Fisher). Monoclonal and polyclonal antibodies, as well as recombinant proteins and calibrator to the several neural specific isoforms of S100β represented by SEQ ID NO's 1-6, are made in-house. Monoclonal and polyclonal GFAP and UCH-L1 antibodies are made in-house or are obtained from Santa Cruz Biotechnology (Santa Cruz, Calif.). Antibodies directed to α-II spectrin and breakdown products, as well as to MAP2, are available from Santa Cruz Biotechnology (Santa Cruz, Calif.). Labels for antibodies of numerous subtypes are available from Invitrogen, Corp. (Carlsbad, Calif.). Protein concentrations in biological samples are determined using bicinchoninic acid microprotein assays (Pierce Inc., Rockford, Ill., USA) with albumin standards. All other necessary reagents and materials are known to those of skill in the art and are readily ascertainable.

### Example 2: Biomarker Assay Development

To determine reactivity specificity of the antibodies to detect a target biomarker, a known quantity of isolated or partially isolated biomarker is analyzed or a tissue panel is probed by western blot. An indirect ELISA is used with the recombinant biomarker protein attached to the ELISA plate to determine optimal concentration of the antibodies used in the assay. Microplate wells are coated with rabbit polyclonal anti-human biomarker antibody. After determining the concentration of rabbit anti-human biomarker antibody for a maximum signal, the lower detection limit of the indirect ELISA for each antibody is determined. An appropriate diluted sample is incubated with a rabbit polyclonal antihuman biomarker antibody for 2 hours and then washed. Biotin labeled monoclonal anti-human biomarker antibody is then added and incubated with captured biomarker. After thorough wash, streptavidin horseradish peroxidase conjugate is added. After 1 hour incubation and the last washing step, the remaining conjugate is allowed to react with substrate of hydrogen peroxide tetramethyl benzadine. The reaction is stopped by addition of the acidic solution, and absorbance of the resulting yellow reaction product is measured at 450 nanometers. The absorbance is proportional to the concentration of the biomarker. A standard curve is constructed by plotting absorbance values as a function of biomarker concentration using calibrator samples, and concentrations of unknown samples are determined using the standard curve.

### Example 3: TBI Patient Samples

Subjects with suspected TBI are enrolled at several investigational sites globally. All Subjects receive standard of care treatment when presenting to the investigational site. Biological samples of blood, urine, saliva, and CSF are collected from the subjects at specified time points. Inclusion criteria for the Subjects include 1) the Subject is at least 18 years of age at screening (has had their 18th birthday) and no more than 80 years of age (did not have their 81st birthday); 2) the Subject received an accelerated or decelerated closed injury to the head (this includes head injuries inflicted by blunt force mechanism) self-reported or witnessed; 3) the biological samples of blood, urine, and saliva are able to be collected within four (4) hours after injury; 4) the Subject is admitted with an initial Glasgow Coma Scale score of 3-8 (severe TBI), or from 5-15 (mild or moderate TBI); 5) the Subject is willing to undergo a computerized tomography (CT) of the head; 6) proper informed consent from patient or guardian. Severe TBI patients may be admitted if in a coma or a vegetative state, while mild to moderate patients may be admitted in a minimally conscious state or suffering from post-traumatic amnesia, retrograde or otherwise. Notwithstanding, the Glasgow Coma Score upon admission to an investigational site shall control which severity of injury the Subject is included. Follow up visits at 7 and 35 days after injury are included in the sample cohort, where again biological samples are drawn. Upon enrollment into the study, further neurocognitive tests such as RBANS, King Devic, GOAT, BESS, and other tests measuring the neurocognitive abilities of a Subject are employed. These tests are also administered during patient follow-up visits to track a patient's recovery and correlate with chronic biomarker measurement.

### Example 4: Stroke Patient Samples

Subjects with suspected stroke are enrolled at several investigational sites globally. All Subjects receive standard of care treatment when presenting to the investigational site. Biological samples of blood, urine, saliva, and CSF are collected from the subjects at specified time points. Inclusion criteria for the Subjects include 1) the Subject is at least 18 years of age at screening (has had their 18th birthday) and no more than 80 years of age (did not have their 81st birthday); 2) the Subject's primary diagnosis is ischemic or hemorrhagic stroke, self-reported or witnessed; 3) the biological samples of blood, urine, and saliva are able to be collected within four (4) hours after injury; 4) the Subject is willing to undergo a computerized tomography (CT) of the head; 5) proper informed consent from patient or guardian.

### Example 5: Normal Patient Samples

Normal Subjects without any known or suspected TBI, liver damage, stroke, or other conditions which may alter protein biomarker levels are enrolled at several investigational sites globally. All Subjects receive standard screening to ensure that no medications or ailments are experienced by the patients prior to enrollment into the study. Biological samples of blood, urine, saliva, and CSF are collected from the subjects upon enrollment. Inclusion criteria for the Subjects include 1) the Subject is at least 18 years of age at screening (has had their 18th birthday) and no more than 80 years of age (did not have their 81st birthday); 2) the Subject is screened and found to not be taking medications or suffering from any neurological injury, neurological disorder, neurotoxicity, or liver injury; 3) proper informed consent from patient or guardian.

### Example 6: Analysis of Mild, Moderate and Severe TBI Markers

Accumulation of the novel neural specific isoforms of S100β, namely S100β2-6, are analyzed in the biological samples, using the processes described herein, through the use of an assay which includes antibodies raised against the peptides of SEQ ID NO 2-6. These assays are then incorporated into the in vitro diagnostic devices where the methods of detection of the neurological condition are performed and the results are illustrated. The assays are for each S100β isoform, but other assays are multiplexed to include one or more combination markers. A portion of the samples are tested using on S100β isoform assays, while a portion tests for a neural specific S100β isoform in combination with glial fibrillary acidic protein (GFAP), a portion tests for a neural specific S100β isoform in combination with Ubiquitin carboxyl-terminal hydrolase L1 (UCH-L1), a portion tests for a neural specific S100β isoform in combination with Microtubule-associated protein 2 (MAP2), a portion tests for a neural specific S100β isoform in combination with myelin basic protein (MBP), a portion tests for a neural specific S100β isoform in combination with α-II spectrin breakdown product (SBDP) that in particular inventive embodiments are SBDP150, SBDP150i SBDP145, SBDP120, a portion tests for a neural specific S100β isoform in combination with neurensin-1 (p24), a portion tests for a neural specific S100β isoform in combination with collapsin response mediated proteins (CRMP's) and breakdown products thereof, a portion tests for a neural specific S100β isoform in combination with synaptotagmin and breakdown products thereof. Normal patient samples are also analyzed for the same biomarkers, and a normal metric is calculated to indicate a non-injury state. The metric is then incorporated into the in vitro diagnostic device either through a computer algorithm, or where a calorimetric indication is provided; the dyes are activated indicating injury when the level of the measured biomarker is higher than what is determined in the normal metric.

Prior to analysis, an assay is developed using a detection and capture antibody, each antibody being specific to the biomarker intended to be measured. For example, for neural specific S100β2 a monoclonal/monoclonal pair (capture/detection) is used to detect the level of biomarkers. Notwithstanding, similar results are achieved through the use of a monoclonal/polyclonal pair, a polyclonal monoclonal pair, and a polyclonal/polyclonal pair. The assay is optimized and tested using a calibrator and spiked serum to ensure that the assay can measure known positive and known negative controls and detect the levels of known proteins within 1 picogram/mL detection sensitivity. The assay is incorporated into an in vitro diagnostic device using a cartridge or other disposable, whereby the cartridge contains the assay and a biological sample collection chamber for receiving the biological sample. The present invention further incorporates by reference the antibody and detection methods for the claimed biomarkers being used in the device for the specific indication disclosed therein presented in US 2007/0003982 A1, US 2005/0260697 A1, US 2009/0317805 A1, US 2005/0260654 A1, US 2009/0087868 A1, US 2010/0317041 A1, US 2011/0177974 A1, US 2010/0047817 A1, US 2012/0196307 A1, US 2011/0082203 A1, US 2011/0097392 A1, US 2011/0143375 A1, US 2013/0029859 A1, US 2012/0202231 A1, and US 2013/0022982 A1 and application Ser. No. 13/470,079. The in vitro diagnostic devices used in this example have incorporated assays contained therein, which assays may be substituted herein using the methods therein contained.

### Example 7: Analysis of Neurotoxic Marker

Accumulation of novel markers indicating neurotoxic insult such as neural specific isoforms of S100β, namely S100β2-6, are analyzed in the biological samples, using the processes described herein, through the use of an assay which includes antibodies raised against the peptides of SEQ ID NO 2-6. These assays are then incorporated into the in vitro diagnostic devices where the methods of detection of the neurological condition are performed and the results are illustrated. The assays are for each S100β isoform, but other assays are multiplexed to include one or more combination markers. A portion of the samples are tested using on S100β isoform assays, while a portion tests for a neural specific S100β isoform in combination with ubiquitin carboxyl-terminal hydrolase-L1 (UCH-L1); spectrin; a spectrin breakdown product (SBDP); MAP1, MAP2; GFAP; ubiquitin carboxyl-terminal esterase; ubiquitin carboxyl-terminal hydrolase; a neuronally-localized intracellular protein; MAP-tau; C-tau; Poly (ADP-ribose) polymerase (PARP); a collapsin response mediator protein; synaptotagmin; βIII-tubulin; S100β; neuron-specific enolase; neurofilament protein light chain; nestin; α-internexin; breakdown products thereof; post-translationally modified forms thereof; derivatives thereof; and combinations thereof; are analyzed in the biological samples taken after TBI using the inventive in vitro diagnostic devices.

Normal patient samples are also analyzed for the same biomarkers, and a normal metric is calculated to indicate a non-injury state. The metric is then incorporated into the in vitro diagnostic device either through a computer algorithm, or in the event of a calorimetric indication, the dyes are activated indicating injury when the level of the measured biomarker is higher than what is determined in the normal metric.

Prior to analysis, an assay is developed using a detection and capture antibody, each antibody being specific to the biomarker intended to be measured. For example, for S100β2, a monoclonal/monoclonal pair (capture/detection) is used to detect the level of biomarkers. Notwithstanding, similar results are achieved through the use of a monoclonal/polyclonal pair, a polyclonal monoclonal pair, and a polyclonal/polyclonal pair. The assay is optimized and tested using a calibrator and spiked serum to ensure that assay can measure known positive and known negative controls and detect the levels of known proteins within 1 picogram/mL detection sensitivity. The assay is incorporated into an in vitro diagnostic device using a cartridge or other disposable, whereby the cartridge contains the assay and a biological sample collection chamber for receiving the biological sample. The present invention further incorporates by reference the antibody and detection methods for the claimed biomarkers being used in the device for the specific indication disclosed therein presented in US 2013/0029362 A1. The in vitro diagnostic devices used in this example have incorporated assays contained therein, which assays may be substituted herein using the methods therein contained.

### Example 8: Severe Traumatic Brain Injury Study

A study was conducted that included 46 human subjects suffering severe traumatic brain injury. Each of these subjects is characterized by being over age 18, having a GCS of less than or equal to 8 and required ventriculostomy and neuromonitoring as part of routine care. A control group A, synonymously detailed as CSF controls, included 10 individuals also being over the age of 18 or older and no injuries. Samples are obtained during spinal anesthesia for routine surgical procedures or access to CSF associated with treatment of hydrocephalus or meningitis. A control group B, synonymously described as normal controls, totaled 64 individuals, each age 18 or older and experiencing multiple injuries without brain injury. Further details with respect to the demographics of the study are provided in Table 1.

TABLE 1
Subject Demographics for Severe Traumatic Brain Injury Study

| | | TBI | CSF Controls | Normal Controls |
|---|---|---|---|---|
| Number | | 46 | 10 | 64 |
| | Males | 34 (73.9%) | 29 (65.9%) | 26 (40.6%) |
| | Females | 12 (26.1%) | 15 (34.1%) | 38 (59.4% |
| Age: | Average | 50.2 | 58.2 1, 2 | 30.09 2, 3 |
| | Std Dev | 19.54 | 20.52 | 15.42 |
| | Minimum | 19 | 23 | 18 |
| | Maximum | 88 | 82 | 74 |
| Race: | Caucasian | 45 | 38 (86.4%) | 52 (81.2%) |
| | Black | 1 | 6 (13.6) | 4 (6.3%) |
| | Asian | | | 7 (10.9%) |
| | Other | | | 1 (1.6%) |

GCS in Emergency Department

| | |
|---|---|
| Average | 5.3 |
| Std Dev | 1.9 |

The level of biomarkers found in the first available CSF and serum samples obtained in the study is provided in the Figures. The average first CSF sample collected as detailed in the Figures was 11.2 hours while the average time for collection of a serum sample subsequent to injury event as per the Figures was 10.1 hours. The quantity of each of the biomarkers of UCH-L1, MAP2, SBDP145, SBDP120, and GFAP is provided for each sample for the cohort of traumatic brain injury sufferers as compared to a control group. The diagnostic utility of the various biomarkers within the first 12 hours subsequent to injury based on a compilation of CSF and serum data is provided in the Figures and indicates in particular the value of GFAP as well as that of additional markers UCH-L1 and the spectrin breakdown products. Elevated levels of UCH-L1 are indicative of the compromise of neuronal cell body damage while an increase in SPDP145 with a corresponding decrease in SBDP120 is suggestive of acute axonal necrosis. The sample analysis was later performed on the cryogenically stored samples obtained for this study and tested for the neural specific isoforms of S100β, namely S100β2-6, where elevated levels were also noted to the same degree.

One subject from the traumatic brain injury cohort was a 52 year old Caucasian woman who had been involved in a motorcycle accident while not wearing a helmet. Upon admission to an emergency room her GCS was 3 and during the first 24 hours subsequent to trauma her best GCS was 8. After 10 days her GCS was 11. CT scanning revealed SAH and facial fractures with a Marshall score of 11 and a Rotterdam score of 2. Ventriculostomy was removed after 5 weeks and an overall good outcome was obtained. Arterial blood pressure (MABP), intracranial pressure (ICP) and cerebral profusion pressure (CPP) for this sufferer of traumatic brain injury as a function of time is depicted in the Figures. A possible secondary insult is noted at approximately 40 hours subsequent to the injury as noted by a drop in MABP and CPP. The changes in concentration of inventive biomarkers per CSF and serum samples from this individual are noted in the Figures. These results, including a sharp increase in GFAP in both the CSF and serum as well as the changes in the other biomarkers depicted in the Figures, provide important clinical information as to the nature of the injury and the types of cells involved, as well as modes of cell death associated with the spectrin breakdown products.

Another individual of the severe traumatic brain injury cohort included a 51 year old Caucasian woman who suffered a crush injury associated with a horse falling on the individual. GCS on admission to emergency room was 3 with imaging analysis initially being unremarkable with minor cortical and subcortical contusions. MRI on day 5 revealed significant contusions in posterior fossa. The Marshall scale at that point was indicated to be 11 with a Rotterdam scale score of 3. The subject deteriorated and care was withdrawn 10 days after injury. The CSF and serum values for this individual during a period of time are provided in the Figures.

Based on the sandwich ELISA testing, S100β2-6 values as a function of time are noted to be markedly elevated relative to normal controls (control group B) as a function of time similar to what is noted for other neuroactive biomarkers such as GFAP and UCH-L1.

The concentration of spectrin breakdown products, MAP2 and UCH-L1 as a function of time subsequent to traumatic brain injury has been reported elsewhere as exemplified in U.S. Pat. Nos. 7,291,710 and 7,396,654 each of which is incorporated herein by reference. In addition, the kinetic profiles of the other stated markers presented herein as one or more additional biomarkers are presented in US 2007/0003982 A1, US 2005/0260697 A1, US 2009/0317805 A1, US 2005/0260654 A1, US 2009/0087868 A1, US 2010/0317041 A1, US 2011/0177974 A1, US 2010/0047817 A1, US 2012/0196307 A1, US 2011/0082203 A1, US 2011/0097392 A1, US 2011/0143375 A1, US 2013/0029859 A1, US 2012/0202231 A1, and US 2013/0022982 A1 and application Ser. No. 13/470,079, each of which is incorporated herein by reference.

An analysis was performed to evaluate the ability of biomarkers measured in serum to predict TBI outcome, specifically GCS. Stepwise regression analysis was the statistical method used to evaluate each of the biomarkers as an independent predictive factor, along with the demographic factors of age and gender, and also interactions between pairs of factors. Interactions determine important predictive potential between related factors, such as when the relationship between a biomarker and outcome may be different for men and women. Such a relationship would be defined as a gender by biomarker interaction.

The resulting analysis identified biomarkers S100β2-6, UCH-L1, MAP2, and GFAP as being statistically significant predictors of GCS. Furthermore, GFAP was shown to have improved predictability when evaluated in interaction with UCH-L1 and gender.

### Example 9

The study of Example 8 is repeated with a moderate traumatic brain injury cohort characterized by GCS scores of between 9 and 11, as well as a mild traumatic brain injury cohort characterized by GCS scores of between 12 and 15. Blood samples are obtained from each patient on arrival to the emergency department of a hospital within 2 hours of injury and measured by ELISA for levels of GFAP in nanograms per milliliter. The results are compared to those of a control group who had not experienced any form of injury. Secondary outcomes included the presence of intracranial lesions in head CT scans.

Over 3 months 53 patients are enrolled: 35 with GCS 13-15, 4 with GCS 9-12 and 14 controls. The mean age was 37 years (range 18-69) and 66% are male. The mean GFAP serum level was 0 in control patients, 0.107 (0.012) in patients with GCS 13-15 and 0.366 (0.126) in GCS 9-12 (P<0.001). The difference between GCS 13-15 and controls was significant at P<0.001. In patients with intracranial lesions on CT, GFAP levels are 0.234 (0.055), compared to 0.085 (0.003) in patients without lesions (P<0.001). There is a significant increase in GFAP in serum following a MTBI compared to uninjured controls in both the mild and moderate groups. GFAP is also significantly associated with the presence of intracranial lesions on CT.

The sample analysis is later performed on the cryogenically stored samples obtained for this study and tested for the neural specific isoforms of S100β, namely S100β2-6, where elevated levels were also noted to the same degree.

The Figures show GFAP concentration for controls as well as individuals in the mild/moderate traumatic brain injury cohort as a function of CT scan results upon admission and 24 hours thereafter. Simultaneous assays are performed in the course of this study for UCH-L1 biomarker. The UCH-L1 concentration derived from the same samples as those used to determine GFAP is provided the Figures. The concentration of UCH-L1 and GFAP, as well as S100β2-6, is provided as a function of injury magnitude between control, mild, and moderate traumatic brain injury as shown in the Figures. The analyses of any of the identified neural specific isoforms, S100β2-6 alone or in combination with one another, or the simultaneous analyses of any neural specific S100β isoform along with UCH-L1 or GFAP from these patients, illustrates the synergistic effect of the inventive process in allowing an investigator to simultaneously diagnose traumatic brain injury as well as discern the level of traumatic brain injury between mild and moderate levels of severity. The Figures show the concentration of the same markers with respect to initial evidence upon hospital admission as to lesions in tomography scans illustrating the high confidence in predictive outcome of the inventive process. The Figures show that both NSE and MAP2 are also elevated in subjects with MTBI in serum both at admission and at 24 hours of follow up. These data demonstrate a synergistic diagnostic effect of measuring multiple biomarkers such as S100β2-6, GFAP, UCH-L1, NSE, and MAP2 in a subject.

Through the simultaneous measurement of multiple biomarkers such as S100β2-6, UCH-L1, GFAP, NSE, and MAP2, rapid and quantifiable determination as to the severity of the brain injury is obtained consistent with GSC scoring and CT scanning, yet in a surprisingly more quantifiable, expeditious, and economic process. Additionally, with a coupled assay for biomarkers indicative of neurological condition, the nature of the neurological abnormality is assessed and, in this particular study, suggestive of neuronal cell body damage. As with severe traumatic brain injury, gender variations are noted suggesting a role for hormonal anti-inflammatories as therapeutic candidates. A receiver operating characteristic (ROC) modeling of S100β2-6, UCH-L1, GFAP, and SBDP145 post TBI further supports the value of simultaneous measurement of these biomarkers, as shown in the Figures.

### Example 10: Detection of Brain Specific or Brain Enriched Isoforms

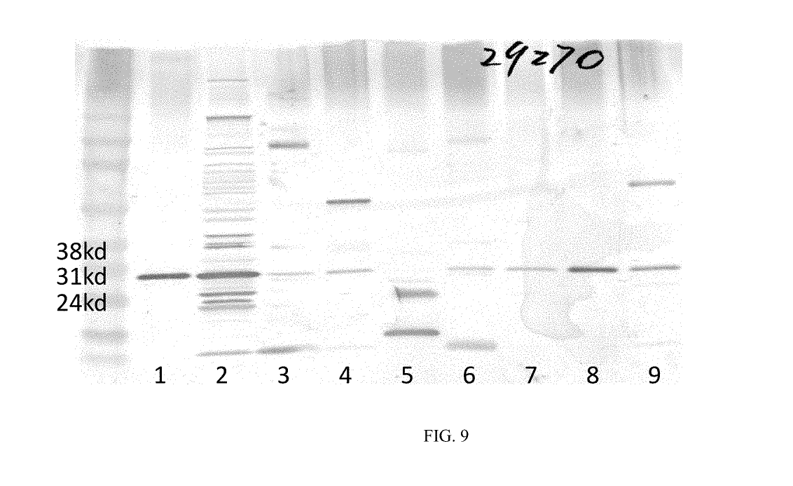

Polyclonal antibodies are raised against the sequences for S100β2 (SEQ ID NO 2). Several biological samples from a human tissue panel are tested with the polyclonal antibodies are raised against the sequences for S100β2. The samples all previously tested strong positive for S100β (now S100β1). The samples are again tested using western blot against the brain specific or brain enriched S100β2. The samples tested include 1) positive control; 2) brain tissue; 3) heart; 4) prostate; 5) skeletal muscle; 6) lung; 7) pancreas; 8) testes; 9) adrenal. The results are illustrated in FIG. 9. The western blots reveal that S100β2 indication is stronger for the brain samples than in the other tissues, providing that the protein is brain enriched.

Similarly, a human blood sample is tested from a patient known to have suffered from TBI. A western blot analysis is performed using a generic S100β antibody. 28 gel spots are chosen from a 2-dimensional electrophoretic gel and subjected to in-gel trypsin digestion. All tryptic digests are analyzed by top 10 data dependent LC-MS/MS. Sequest search is validated by Scaffold and fragments from peptide +RRSCKKADSKGLQPSRS (S100β2) (SEQ ID NO 7) are detected indicated this presence of this brain enriched isoform in blood is proportional to the extent of injury.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

Sequence Listing

SEQ ID NO 1:
MSELEKAMVALIDVFHQYSGREGDKHKLKKSELKELINNELSHFLEEIKE
QEVVDKVMETLDNDGDGECDFQEFMAFVAMVTTACHEFFEHE
SEQ ID NO 2:
MSELEKAMVALIDVFHQYSGREGDKHKLKKSELKELINNELSHFLEEIKE
QEVVDKVMETLDNDGDGECDFQEFMAFVAMRRSCKKADSKGLQPSRS
SEQ ID NO 3:
MSELEKAMRRSCKKADSKGLQPSRS
SEQ ID NO 4:
MSELEKAMEIKEQEVVDKVMETLDNDGDGECDFQEFMAFVAMVTTACHEF
FEHE
SEQ ID NO 5:
MSELEKAMEIKEQEVVDKVMETLDNDGDGECDFQEFMAFVAMRRSCKKAD
SKGLQPSRS
SEQ ID NO 6:
MSELEKAMVALIDVFHQYSGREGDKHKLKKSELKELINNELSHFLEEIKE
QERRSCKKADSKGLQPSRS
SEQ ID NO 7:
RRSCKKADSKGLQPSRS

1. An in vitro diagnostic device for detecting a neural injury or neuronal disorder in a subject, the device comprising:
a sample chamber for holding a first biological sample collected from the subject;
an assay module in fluid communication with said sample chamber, said assay module containing an agent for detecting one or more biomarkers of a neural injury or neuronal disorder selected from the group consisting of S100β2, S100β3, S100β4, S100β5, or S100β6;
wherein said assay module analyzes the first biological sample to detect the amount of the one or more biomarker present in said sample;
a user interface, wherein said user interface relates the amount of the one or more biomarker measured in the assay module to detecting a neural injury or neuronal disorder in the subject or the severity of neural injury or neuronal disorder in the subject.

2. The device of claim 1, wherein the neural injury or neuronal disorder is one of: stroke, epilepsy, hypoxic ischemic encephalopathy (HIE), chronic traumatic encephalopathy (CTE), Alzheimer's disease (AD), Parkinson's disease (PD), traumatic brain injury (TBI), neurotoxicity, spinal cord injury (SCI), or neural cell damage.

3. The device of claim 1 wherein said assay module further comprises at least one additional agent selective to measure for at least one additional biomarker selected from the group consisting of: glial fibrillary acid protein (GFAP) or one or more GFAP breakdown products that correlates with a quantity of said GFAP; Ubiquitin carboxyl-terminal hydrolase L1 (UCH-L1); neuron specific enolase (NSE); Microtubule-associated protein 2 (MAP2); myelin basic protein (MBP); α-II spectrin breakdown product (SBDP) of at least one of SBDP150, SBDP150i SBDP145, SBDP120, and S100β1; vesicular membrane protein neurensin-1 (p24); collapsin response mediated proteins (CRMP's) or one or more breakdown products that correlates with a quantity of one of said CRMPs; and synaptotagmin or one or more breakdown products that correlates with a quantity of said synaptotagmin.

4. The device of claim 1 wherein the first biological sample is selected from the group consisting of blood, blood plasma, serum, sweat, saliva, cerebrospinal fluid (CSF), and urine.

5. The device of claim 1 wherein said assay further comprises a dye providing a colorimetric change in response to the one or more biomarker present in the first biological sample.

6. The device of claim 1 wherein said assay module is an immunoassay.

7. The device of claim 6 wherein the immunoassay is an ELISA.

8. The device of claim 1 wherein said agent is an antibody or a protein.

9. The device of claim 1 further comprising a power supply and a data processing module in operable communication with said power supply and said assay module wherein said data processing module has an output the relates to detecting the neural injury or neuronal disorder in the subject; the output displaying the amount of the one or more biomarker measured in said sample; the output displaying the presence or absence of a neural injury or neuronal disorder; or the output displaying the severity of neural injury or neuronal disorder.

10. The device of claim 9, further comprising analysis of a second biological sample obtained from the subject, at some time after the first sample is collected, wherein if the device detects a decreased amount of the one or more biomarker in the second sample relative to the first sample, a recovery output is provided by the data processing module.

11. The device of claim 9 further comprising a display in electrical communication with said data processing module and displaying the output as at least one of an amount of the one or more biomarker, a comparison between the amount of the one or more biomarker and a control, presence of the neural injury or neuronal disorder, or severity of the neural injury or neuronal disorder.

12. The device of claim 9 further comprising a transponder for communication with a remote device.

13. The device of claim 9 wherein the communication is digital.

14. A method for using an in vitro diagnostic device for detecting a neural injury or neuronal disorder in a subject, the method comprising:
calibrating an in vitro diagnostic device incorporating an assay for measuring one or more biomarkers of a neural injury or neuronal disorder in a biological sample, the one or more biomarkers selected from the group consisting of S100β2, S100β3, S100β4, S100β5, or S100β6, or breakdown products thereof;
obtaining a biological sample from a subject;
applying said sample to said in vitro diagnostic device wherein said assay includes reagents to determine the amount of the one or more biomarker present in said sample, wherein said device provides an output which relates the amount of the one or more biomarker detected to a neural injury or neuronal disorder, or lack thereof, in the subject.

15. The method of claim 14 further comprising:
calibrating an in vitro diagnostic device incorporating an assay for additionally measuring at least one additional biomarker selected from the group consisting of: (GFAP) or one of its breakdown products, Ubiquitin carboxyl-terminal hydrolase L1 (UCH-L1), neuron specific enolase (NSE), Microtubule-associated protein 2 (MAP2), myelin basic protein (MBP), α-II spectrin breakdown product (SBDP) that in particular inventive embodiments are SBDP150, SBDP150i SBDP145, SBDP120, S100β1, vesicular membrane protein neurensin-1 (p24), collapsin response mediated proteins (CRMP's) and breakdown products thereof, or synaptotagmin and breakdown products thereof;
applying said sample to said in vitro diagnostic device wherein said assay includes reagents to determine the amount of the additional biomarker present in said sample, wherein said device provides an output which relates the amount of the additional biomarker detected, alone or in synergistic combination with the one or more biomarker, to a neural injury or neuronal disorder, or lack thereof, in the subject.

16. A method of treating a neural injury or neuronal disorder in a subject:
calibrating an in vitro diagnostic device incorporating an assay for measuring for one or more biomarkers in a biological sample, the one or more biomarkers selected from the group consisting of S100β2, S100β3, S100β4, S100β5, or S100β6 and breakdown products thereof;
obtaining a biological sample from a subject;
applying said sample to said in vitro diagnostic device wherein said assay includes reagents to determine the amount of the one or more biomarker present in said sample, wherein said device provides an output which relates the amount of the one or more biomarker detected to a neural injury or neuronal disorder, or lack thereof, in the subject, wherein if said output of said in vitro diagnostic device relates the amount of the one or more biomarker to a neuronal injury or neuronal disorder a therapeutic intervention is employed to treat injury and/or inhibit injury progression.
